(19) United States
(12) Patent Application Publication
(10) Pub. No.: US 2019/0060661 A1
(43) Pub. Date: Feb. 28, 2019

(54) METHOD FOR IRRADIATING SKIN AND DEEPER TISSUES USING UNPACKAGED LED LIGHTS TO IMPROVE SKIN AND TISSUE ABSORPTION OF LOW LEVEL LIGHT ENERGY

(71) Applicants: William Fred Wiedemann, III, Livermore, CA (US); Linda Rayburn, San Clemente, CA (US); Eric Philip Wiedemann, Livermore, CA (US); William Fred Wiedemann, Jr., Carlsbad, CA (US)

(72) Inventors: William Fred Wiedemann, III, Livermore, CA (US); Linda Rayburn, San Clemente, CA (US); Eric Philip Wiedemann, Livermore, CA (US); William Fred Wiedemann, Jr., Carlsbad, CA (US)

(21) Appl. No.: 16/116,111

(22) Filed: Aug. 29, 2018

Related U.S. Application Data
(60) Provisional application No. 62/551,428, filed on Aug. 29, 2017.

(51) Int. Cl. A61N 5/06

(52) U.S. Cl. CPC A61N 5/0616; A61B 2018/00452; A61N 2005/0643; A61N 2005/0651

(57) ABSTRACT

A method for irradiating skin and deeper tissue using unpackaged LED lights to improve skin and tissue absorption of low level light energy may be provided. The apparatus embodied by the method may be formed by selectively mounting chip-on-board LEDs in various array so that the skin gets irradiated from all angles. The LEDs may be associated with a heat sink provided by the substrate of PCB to more effectively dissipate its heat, allowing the apparatus to run at higher outputs.

### CROSS-REFERENCE TO RELATED APPLICATION

This application claims the benefit of priority of U.S. provisional application No. 62/551,428, filed 29 Aug. 2017, the contents of which are herein incorporated by reference.

### BACKGROUND OF THE INVENTION

The present invention relates to overall wellness and skin rejuvenation and, more particularly, to a method for irradiating skin and deeper tissue using unpackaged LED lights to improve skin and tissue absorption of low level light energy.

On the treatment tables of medical spas and facial cleaner practitioners, patients are routinely treated with output light to rejuvenate their skin and promote overall wellness. Current treatment methods utilize packaged LEDs as the output light. These light-emitting devices tend to limit the usable portion of the output light to a narrow beam, requiring the practitioner to continuously move the output light in order to effectively apply the treatment. Such configurations of LEDs limit the irradiation to a number of focused beams, reducing overall efficiency especially in the amount of time the treatment takes.

Furthermore, the packaged LEDs currently used limit the light output because their heat cannot be dissipated. Therefore, the current must be lower and the output light energy is lower.

As can be seen, there is a need for a method for irradiating skin and deeper tissue using unpackaged LED lights to improve skin and tissue absorption of low level light energy. By using unpackaged LEDs—Chip on Board (COB) LEDs—more output power can be achieved as the LED chip can more easily dissipate heat directly to a heat sink on which the LED chip is mounted. With more output light, the LED does not have to be focused to be effective. Furthermore, irradiating the skin with light from all directions enables the skin cells and tissue to react more effectively to the irradiated energy and allows the distance between the LEDs and the skin to be treated to vary without resulting in the normal degradation of energy as the distance is increased. This benefit comes over the first few inches or from the array of LEDs because as the distance increases the energy from additional LEDs reaches the skin.

By eliminating the LED mounting package and by mounting the LED chip directly on the PCB or substrate, the LED chip can be powered with more current and therefore put out more light energy. As the LED is putting out more light energy, the LED can be used without the need to focus the light. Since the light is not focused it can irradiate the skin cells and deeper tissue from all directions, as the power does not degrade from change in direction as it would if using a narrow-beam light source. Simple parabolic focusing can be used with the technology to focus the light without the need for the COB LED technology to be packaged into a plastic or glass lens as current packaged LEDs are. This parabolic cone helps to steer the light. It does not focus it to a narrow beam.

In short, by not packaging the LED chips, the LEDs can dissipate their heat better and so be driven at higher currents—i.e., increased light output. Moreover, the unpackaged LEDs can be selectively mounted on a substrate to shape a radiation pattern, and produce wider array of angles—improving the tissue absorption of the low-level light energy.

By using an unpackaged LED chip, the LED chip can be mounted directly on a heatsink. As the LED chip can now more effectively dissipate its heat it can run at higher outputs. As it can run at higher outputs it can be used without a focusing mechanism. Without a focusing mechanism the resulting product is thinner (thus, more flexible) and so can be used in wider variety of applications.

### SUMMARY OF THE INVENTION

In one aspect of the present invention, a method for rejuvenating skin, includes the steps of: providing a plurality of unpackaged LED lights; positioning the unpackaged LED lights adjacent to a portion of skin to rejuvenate; and powering the unpackaged LED lights so as to irradiate said portion of skin from a plurality of angles

In another aspect of the present invention, the method for rejuvenating skin, includes the steps of: providing a plurality of unpackaged LED lights in a chip-on-board assembly, wherein the plurality of unpackaged LED lights is mounted on a flexible substrate so as to be operatively associated with a heat sink thereon; positioning the unpackaged LED lights adjacent to a portion of skin to rejuvenate; and powering the unpackaged LED lights so as to irradiate said portion of skin from a plurality of angles, wherein the flexible substrate may provide one or more handles, wherein the plurality of unpackaged LED lights may be arranged in a grid, and wherein the plurality of unpackaged LED lights is not associated with a focusing mechanism.

Additionally to facilitate manufacturing a large array of LEDs, each LED can be on its own small printed circuit board and then used as a subassembly for a very large array. This can improve manufacturability and manufacturing yield of the large array.

These and other features, aspects and advantages of the present invention will become better understood with reference to the following drawings, description and claims.

### BRIEF DESCRIPTION OF THE DRAWINGS

FIG. 1 is an elevational view of an exemplary embodiment of the present invention, illustrating a substrate with a plurality of COB LEDs;

FIG. 2 is an elevational view of an exemplary embodiment of the present invention, illustrating a flexible printed circuit board with COB array; and

FIG. 3 is an enlarged elevational view of surface mounted COB LED of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method for irradiating skin and deeper tissue using unpackaged LED lights to improve skin and tissue absorption of low level light energy. The apparatus embodied by the method may be formed by selectively mounting chip-on-board LEDs in various array so that the skin gets irradiated from all angles. The LEDs may be associated with a heat sink provided by the substrate of PCB to more effectively dissipate its heat, allowing the apparatus to run at higher outputs.

The present invention may include COB LED Chips carefully mounted to a rigid or flexible heatsink. The LED Chips may be electrically interconnected in a parallel and serial configuration to optimize their performance and eliminate the need for resistors when connecting to power sources with different voltages and or currents. LEDs are typically small (less than 1 mm) and so can be used to shape a radiation pattern through selectively laying out a grid pattern. An optional parabolic cone may be provided to steer the output light.

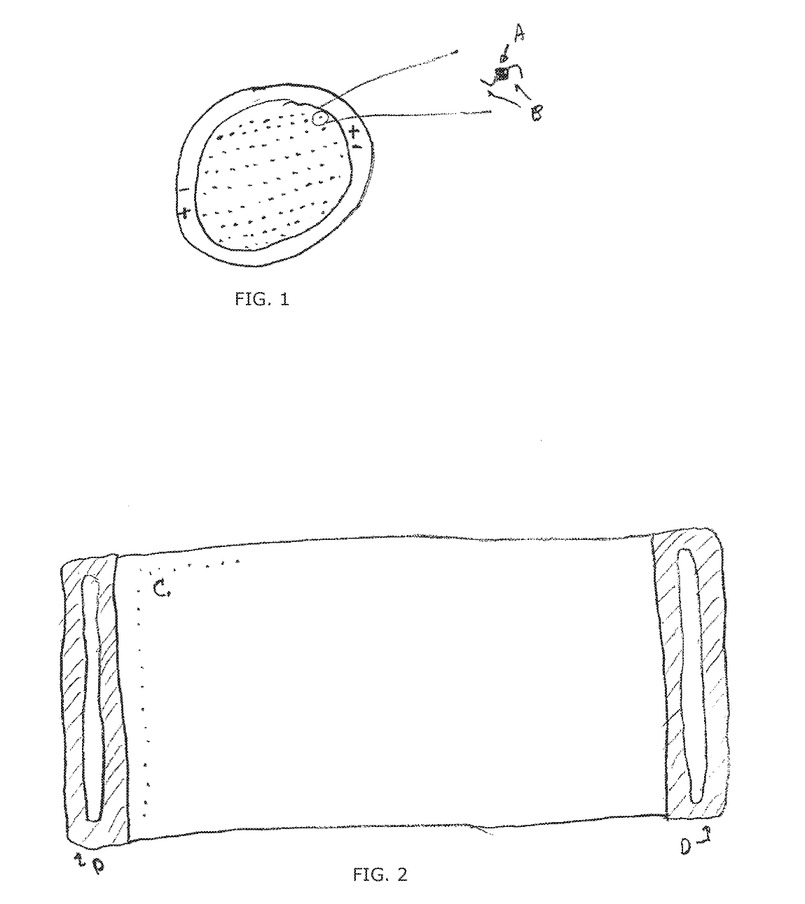

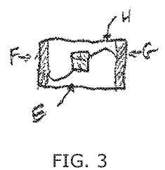

Referring to FIG. 1, substrates such as aluminum discs may be mounted with COB LEDs chips (A) electrically connected to the substrate via wire bonds to plus and minus traces (B) on Printed Circuit Board (PCB). As illustrated in FIG. 2, the flexible PCB may be mounted with various arrays (C) of COB LEDs, such as a 1500 (30 parallel by 50 series) configuration. Handles (D) may be attached to the PCB to manipulate the PCB during treatment, e.g., to position the bendable module over the face and body parts of a patient. FIG. 3 is an enlarged view of illustrating a surface mounted COB LED (approximately 2 mm by 1 mm) including COB die/chip (E), a plus connection (F), a minus connection (G), and a wire bond (H). The user would connect the COB LEDs in an optimized serial and parallel configuration to eliminate energy that is dissipated in the resistors. Furthermore, regardless of the size of the configuration the PCB may have only one circuit and two contacts.

A method of using the present invention may include the following. The LED mounted COB disclosed above may be provided. A user may use the COB LEDs to make rejuvenate skin, reduce pain and tissue inflammation through a lighter weight and more flexible applicator—a simple COB. The COB may be held about half an inch from the skin of the patient. The flexibility of the COB further affords greater applications, for example to portions of a patient's body that are currently not being treated.

Additionally, the present invention may be used in small handheld devices to provide professional grade skin irradiation, pain reduction, and tissue inflammation systems.

It should be understood, of course, that the foregoing relates to exemplary embodiments of the invention and that modifications may be made without departing from the spirit and scope of the invention as set forth in the following claims.

What is claimed is:

1. A method for rejuvenating skin, comprising the steps of:
providing a plurality of unpackaged LED lights;
positioning the unpackaged LED lights adjacent to a portion of skin to rejuvenate; and
powering the unpackaged LED lights so as to irradiate said portion of skin from a plurality of angles.

2. The method of claim 1, furthering providing the plurality of unpackaged LED lights in a chip-on-board assembly.

3. The method of claim 1, wherein the plurality of unpackaged LED lights is not associated with a focusing mechanism.

4. The method of claim 1, wherein the plurality of unpackaged LED lights is mounted on a flexible substrate.

5. The method of claim 1, wherein the plurality of unpackaged LED lights is mounted on a flexible PCB.

6. The method of claim 1, wherein the plurality of unpackaged LED lights is operatively associated with a heat sink.

7. The method of claim 4, wherein the flexible substrate provides one or more handles.

8. The method of claim 4, wherein the plurality of unpackaged LED lights is arranged in a grid.

9. A method for rejuvenating skin, comprising the steps of:
providing a plurality of unpackaged LED lights in a chip-on-board assembly, wherein the plurality of unpackaged LED lights is mounted on a flexible substrate so as to be operatively associated with a heat sink thereon;
positioning the unpackaged LED lights adjacent to a portion of skin to rejuvenate; and
powering the unpackaged LED lights so as to irradiate said portion of skin from a plurality of angles.

10. The method of claim 9, wherein the flexible substrate provides one or more handles.

11. The method of claim 9, wherein the plurality of unpackaged LED lights is arranged in a grid.

12. The method of claim 9, wherein the plurality of unpackaged LED lights is not associated with a focusing mechanism.
